(19) United States
(12) Patent Application Publication
(10) Pub. No.: US 2022/0211746 A1
(43) Pub. Date: Jul. 7, 2022

(54) SPECIAL HEPARINOID COMPOSITION, WITH REPAIR, PROPHYLACTIC AND ANTI-INFLAMMATORY EFFECTS, APPLIED TO THE LUNGS

(71) Applicant: BIONOVAX CORP., Taoyuan (TW)

(72) Inventor: Shean-Ren CHUU, Taoyuan (TW)

(21) Appl. No.: 17/143,683

(22) Filed: Jan. 7, 2021

(51) Int. Cl. A61K 31/727; A61K 9/00

(52) U.S. Cl. CPC A61K 31/727; A61K 9/008; A61K 9/0043

(57) ABSTRACT

The present invention provides a special heparinoid composition, with repair, prophylactic and anti-inflammatory effects, applied to the lungs, which includes at least two glycosaminoglycans, which contain 5˜95 wt % of macromolecular heparin sodium, 3˜40 wt % of low-molecule-weight heparin sodium and 5˜95 wt % of dermatan sulfate are formulated into a solution with a mixture to water ratio of 0.1˜0.6% by weight, and a delivery device is connected to the patient's nose or mouth to deliver to the lungs. The composition of this type of heparinoid has an appropriate dose of disaccharide NH— or O— sulfo and total sulfates, which can make the blood have an anticoagulant effect. During the prophylactic period, more heparin sodium can be added for increasing oxygen and nutrient delivery. At repairing period, more dermatan sulfate with or without chondroitin sulfate can be the main body for better anti-inflammatory repair.

### BACKGROUND OF THE INVENTION

### 1. Field of the Invention

The present invention relates to a special heparinoid composition, with repair, prophylactic and anti-inflammatory effects, applied to the lungs, more particularly, the heparinoid composed of a special formula is used as the main body of anticoagulation, and is delivered via respiratory system to the lungs with a special delivery device, which can balance and achieve anticoagulation that is not easy to cause bleeding risk while having a special anti-inflammatory effect.

### 2. Description of the Related Art

Coronavirus (CoV) belongs to a large group of viruses, which can infect humans and vertebrates and cause respiratory diseases. The main symptoms of human infection with coronavirus are respiratory symptoms. Mild symptoms are similar to the common cold, including nasal congestion, runny nose, cough, fever and other general symptoms of upper respiratory tract infection. However, after infection of severe acute respiratory syndrome coronavirus (SARS-CoV), middle east respiratory syndrome coronavirus (MERS-CoV), and severe special infectious pneumonia (commonly known as novel coronavirus (COVID-19) pneumonia, referred to as new coronary pneumonia (SARS-CoV-2), the symptoms of coronavirus are more severe than those of ordinary humans, and severe symptoms of acute lower respiratory tract infections such as severe pneumonia and respiratory failure may occur.

In patients with a new type of coronavirus infection that progresses from mild to severe, chest X-rays will have infiltrates on both lungs, or computer tomography (CT) will have viral pneumonia manifestations of ground glass lesions (GGO). The basic pathological causes are acute respiratory distress syndrome (ARDS), and respiratory failure causes oxygenation disorders, septic shock, metabolic acidosis or coagulopathy, etc., and even death. From a pathological point of view, it is mainly due to the deterioration of the alveolar epithelial barrier, the increased permeability of pulmonary microvascular endothelium, excessive immune cell infiltration, and excessive secretion of inflammatory substances, which will cause diffuse acute lung injury, that is, a severe inflammatory response is diffused with fibrin deposited in capillaries and alveoli. In short, the gas in the alveoli and its capillaries cannot be exchanged, and a large number of immune thrombosis will be formed, leading to lung failure, and the lung will become a dead space without respiratory function. In addition, the current treatment of acute respiratory distress syndrome is still mainly supportive treatment, including mechanical ventilation with respirators, oxygen therapy, the use of neuromuscular blockers or antibiotics/antiviral drugs, and infusion therapy.

Furthermore, the clinical and pathological features of the aforementioned acute respiratory distress syndrome are mainly activation of coagulation and excessive inflammation. Among them, the increased permeability of the pulmonary microvascular endothelium will cause the new coronary pneumonia virus to enter the cell through the ACE-2 (Angiotensin Converting Enzyme 2) receptor on the cell surface, thereby causing human infection. For example, the human body's ACE-2 activity will be significantly reduced due to the new coronavirus infection, and cause a significant increase in ACE-1, Ang II (angiotensin II), followed by pulmonary edema, severe lung injury and acute lung exhaustion, etc., in addition to the invasion of the virus, severe symptoms often appear systemic inflammatory reactions, septic shock and other problems.

In most patients with severe new coronary pneumonia, it will be accompanied by systemic coagulation abnormalities, such as blood clots caused by blood clotting in the lungs, heart, liver, and kidneys. The cause of coagulation is that the new crown pneumonia virus can bind to ACE-2 on endothelial cells and damage the blood circulation of blood vessels, and then there will be reactive thrombocytosis, which causes the coagulation factors in the blood to be continuously activated and thrombin is generated. It converts fibrinogen into fibrin, which is diffusely deposited in alveolar and interstitial cells, and even the mass formation of diffuse microthrombi causes systemic inflammatory reactions, septic shock, various organ failures, etc. In the human immune system, innate immunity is the first line of defense against the invasion of foreign pathogens. When the virus invades the lungs or other organs of the human body, it will call up immune cells to the infected site to initiate an inflammatory response, and quickly clear the pathogenic bacteria to play a protective role. When the load of natural immunity is too much, it will stimulate the acquired immune system (adaptive immunity) to activate specific T cells and B cells (including macrophages, monocytes, dendritic cells, neutrophils and epithelial cells, etc.). However, an overreaction of the immune system will cause the destruction of the vascular endothelium, and the cascade of coagulation factors in the blood will be continuously activated to cause excessive inflammation, and then cause immune thrombosis.

The immune thrombosis mentioned above has two main functions, protecting the integrity of endothelial cells. If it is damaged, it will produce microthrombi to initiate coagulation and cause inflammation to proceed (In the inflammatory period, destruction is more; but in the late stage of inflammation, repair is the main task. The effects of inflammation and coagulation are actually to protect the integrity of endothelial cells). For example, extensive endothelial destruction will expose and release the collagen under the endothelium and release tissue factors (TF), namely FIII, which is related to blood coagulation and inflammation.

There is a receptor for the coagulation factor FV at the N-terminus of the FIII molecule, which can combine with the coagulation factor FVII in the plasma to form an active form of TF-FVIIa, and then strengthen the coagulation factors FX, FXa and FVa to form prothrombinase that causes blood clotting prothrombin to be converted into thrombin, which contains a large amount of fibrin and microthrombi, which plays an important role in the process of blood clotting and is important for cell repair, providing many immune cells to attach, convene and activate.

In addition, a report published in the Journal of the American College of Cardiology (JACC) showed that the use of anticoagulant drugs (including oral antithrombotic drugs, subcutaneous heparin and intravenous heparin, blood thinners that slow blood clotting) can improve hospitalization. Regardless of whether patients in the intensive care unit (ICU), after receiving anticoagulant drugs, the results show that they have improved, and the survival rate of patients has improved. In addition, patients who received anticoagulant drugs had higher levels of inflammatory markers than those who did not receive anticoagulant drug treatment, suggesting that patients with more severe disease can benefit from anticoagulant drugs early.

The macromolecular heparin sodium, with many NH— or O— sulfo ends, by virtue of its own highly negative charged property, can quickly attract the coronavirus prominent protein spikes, then wrap up the virus to prevent the spread of the virus, and eventually kill the virus before it can enter the human cells. This unique action quickly quenches the development and prevents the spread of virus and this special mechanism can be used to eliminate the coronavirus prior to the onset of disease.

Anticoagulant drugs currently in clinical use include unfractionated heparin (UFH) and low molecular weight heparin (LMWH). Since the anticoagulant and antithrombotic activity of heparin is related to the molecular weight, unfractionated heparin has the risk of bleeding in clinical use, and its clinical application is limited. Therefore, with the in-depth clinical and pharmacological research of heparin, low molecular weight heparin is mainly used. Compared with unfractionated heparin, low molecular weight heparin has better antithrombotic effect, and unfractionated heparin can only be administered by continuous intravenous infusion or subcutaneous administration. Therefore, using a special heparinoid composed of a special effective formula as the main body of anticoagulation and supplement with a special delivery device to transport to the lungs to effectively increase the absorption rate and to achieve the purpose of coagulation along with repairing, prophylactic and anti-inflammatory effects has been the focus of this invention.

### SUMMARY OF THE INVENTION

Therefore, in view of the above-mentioned deficiencies, the inventor collected relevant information, after multiple evaluations and considerations, and continued trial and modification based on years of research and development experience accumulated in this field and began to design the formula composition of this special heparinoid composition supplemented by a special delivery device to improve the absorption rate of the lungs and the convenience of use by patients.

It is therefore the main object of the present invention to provide a special heparinoid composition, with repair, prophylactic and anti-inflammatory effects, applied to the lungs, which includes at least two glycosaminoglycans, which contain 5˜95 wt % of macromolecular heparin sodium, 3˜40 wt % of low-molecule-weight heparin sodium and 5˜95 wt % of dermatan sulfate are formulated into a solution with a mixture to water ratio of 0.1˜0.6% by weight, and a delivery device is connected to the patient's nose or mouth to deliver to the lungs. The composition of this type of heparinoid has an appropriate dose of disaccharide NH— or O— sulfo and total sulfates, which can make the blood have an anticoagulant effect. During the prophylactic period, more heparin can be the main body to promote blood circulation, increase oxygen and nutrient delivery capability. During the repair period, if potential bleeding is considered, heparin can be reduced appropriately, but at the same time, the proportion of dermatan sulfate and chondroitin sulfate with anti-inflammatory effects is added to balance the anticoagulation action that has a lower risk to cause bleeding while having a special anti-inflammatory effect.

It is another objective of the present invention to provide a special heparinoid composition, with repair, prophylactic and anti-inflammatory effects, applied to the lungs, wherein the heparinoid composed of a special formula can be formulated into a dosage form suitable for use in the nose or respiratory tract of patients, and a delivery device such as ultrasonic nebulizer, sprayer, etc. is used to deliver the heparinoid to the lungs through oral or nasal administration routes directly by spray or inhalation, thereby improving the absorption rate and convenience of use in the lungs.

### DETAILED DESCRIPTION OF THE PREFERRED EMBODIMENT

The present invention provides a special heparinoid composition, with repair, prophylactic and anti-inflammatory effects, via respiratory system applied to the lungs, which includes at least two glycosaminoglycans. These glycosaminoglycans include, but not limited to, 5˜95% of macromolecular heparin sodium, 3˜20 wt % of low-molecule-weight heparin sodium (LMWH Sodium) and 5˜95 wt % dermatan sulfate (DS). Dermatan sulfate can also be mixed with 0˜15 wt % chondroitin sulfate (CS), because chondroitin sulfate B contains iduronic acid instead of D-glucuronic acid, similar in composition to dermatan sulfate. In this embodiment, the weight-average molecular weight of macromolecular heparin sodium (Weight-average molecular weight, hereinafter referred to as Mw) is between 5˜40 kilo Daltons (kilo Daltons, hereinafter referred to as kDa), preferably about 16 kDa; the weight-average molecular weight of low-molecule-weight heparin sodium is between 0.4˜15 kDa, preferably about 4.4 kDa; the weight-average molecular weight of dermatan sulfate is between 5˜80 kDa, preferably about 30 kDa; the weight-average molecular weight of chondroitin sulfate is between 5˜50 kDa, preferably about 15 kDa. It is understandable that in order to distinguish between the macromolecular heparin and the low-molecule-weight heparin mentioned in the present invention (including sodium heparin, heparin calcium, etc.), the weight-average molecular weight (Mw) of traditional heparin (i.e., unfractionated heparin) is about 15 kDa, most of the weight-average molecular weight is above 5.4 kDa. In this embodiment, heparin with a weight-average molecular weight above 6 kDa can be defined as a macromolecular heparin, and heparin with a weight-average molecular weight below 6 kDa can be defined as low-molecule-weight heparin, but in actual distinction, this is not a limitation.

In this embodiment, the source of heparin used is a sulfated glycosaminoglycan (including heparin large or small molecules, dermatan sulfate, sodium salt of chondroitin sulfate, etc.) extracted from the small intestinal mucosa or organ tissues of natural animals (such as cows, pigs, chickens or sharks), or chondroitin sulfate and dermatan sulfate, which are composed of galactose and glucuronic acid extracted from animal cartilage tissues (such as nasal bones, throat bones and trachea, etc.). Then mix a certain proportion of macromolecular heparin and low-molecule-weight heparin to produce heparin with a specific molecular weight, dermatan sulfate and/or a mixture without chondroitin sulfate (i.e., heparin), as the main body of anticoagulation, and dissolve in pure water or deionized water into a 0.1˜0.6% solution (wt/wt, that is, the weight ratio of the mixture to water). A special delivery device is used to deliver the heparin composition from the nose or mouth of the patient to the lungs to help the lungs fight blood clotting and to achieve the purpose of anti-inflammatory, repair, and health care simultaneously. After the preparation of the heparinoid composed of the above special formula is completed, the index data can also be formulated according to its required remedial characteristics. The expected indicators include component verification analysis, solubility, pH tolerance, temperature and humidity tolerance, light tolerance, and reversible recovery of activity, stability of organic solvents, etc. It is supplemented by precise instruments for effective monitoring and scientific data acquisition, as a reference for future process control.

The heparinoid of Example 1 has 85 wt % by weight of macromolecular heparin sodium, 10 wt % by weight of low-molecule-weight heparin sodium and 5 wt % by weight of dermatan sulfate, and is formulated into a 0.2% solution, which can be used alone or in combination with pharmacologically acceptable excipients (such as preservatives, glycerin, peppermint, etc.) to make a spray or solution dosage form. The heparinoid can be applied to a special delivery device that can deliver the heparinoid from the patient's nose or mouth to the lungs. Different ratios of heparin can also be used during the prophylactic and repair period to control the desired effect. During the prophylactic period, more heparin can be the main body to promote blood circulation, increase oxygen and nutrient delivery capability. During the repair period, if potential bleeding is considered, heparin can be reduced appropriately, but at the same time, the proportion of dermatan sulfate and chondroitin sulfate with anti-inflammatory effects is added to balance the anticoagulation that has a lower risk to cause bleeding while having a special anti-inflammatory effect.

In the following embodiments, only the proportions of the above-mentioned heparin main components used are explained. The heparinoid of Example 2 has 10 wt % of macromolecular heparin sodium, 40 wt % of low-molecule-weight heparin sodium, and 50 wt % of dermatan sulfate, and is formulated into a 0.2% solution. The heparinoid of Example 3 has 10 wt % of macromolecular heparin sodium, 5 wt % of low-molecule-weight heparin sodium, and 85 wt % of dermatan sulfate, and is formulated into a 0.3% solution. The heparinoid of Example 4 has 80 wt % of macromolecular heparin sodium, 10 wt % of low-molecule-weight heparin sodium, and 10 wt % of dermatan sulfate, and is formulated into a 0.3% solution. The heparinoid of Example 5 has 7 wt % of macromolecular heparin sodium, 3 wt % of low-molecule-weight heparin sodium, and 90 wt % of dermatan sulfate, and is formulated into a 0.2% solution. The heparinoid of Example 6 has 7 wt % of macromolecular heparin sodium, 3 wt % of low-molecule-weight heparin sodium, 88 wt % of dermatan sulfate, and 2 wt % of chondroitin sulfate, and is formulated into a 0.2% solution. The composition ratio, combination and principle of the heparin with this special formula can be adjusted appropriately according to the actual effect required, including the prevention and repair of the lung at different periods, and it can also be applied to other medical applications, such as medical care and repair for wound healing and cosmetic effects. All simple modifications and equivalent changes of heparinoid using the contents of the specification of the present invention should be included in the patent scope of the present invention in the same way.

The special delivery devices applicable to the heparinoid mentioned above include ultrasonic nebulizers, sprayers, vaporizers, etc., but it is not limited to this. You can also use metered dose inhalers (MDIs), nasal inhalers and other inhalation delivery devices. When heparin is formulated into an oral spray or nasal spray for administration, a spray delivery device can be used one or more times a day at a certain dose of about 35kIU (International Unit) for indoor use. When heparin is formulated as an inhalant for administration, a nasal spray inhaled delivery device can be used at a certain dose of about 250˜750IU for use when going out. The invention allows patients to use spray or inhalation delivery methods to directly deliver special formula heparinoids to the lungs through the nose or oral cavity to increase the rate of absorption by the lungs and the convenience of use.

The heparinoid composed of the above-mentioned special formula of the present invention is made based on at least two glycosaminoglycans, with appropriate disaccharide NH— or O— sulfo and total sulfates and is connected to a special delivery device and is delivered to the lungs from the patient's oral or nasal route of administration.

Since heparinoids are absorbed from the lungs from the bronchus into epithelial cells and alveoli, a small number of them enter the venous blood vessels, which can inhibit the coagulation factors and make them inactivated, while avoiding the production of thrombin, which can reduce fibrin deposition in tissues and cell proliferation related to fibrinolytic enzymes, etc., to help lung anticoagulation. Through the conversion of glycosaminoglycan stem cells to differentiate into cells with specific functions, it can make the blood have a better anticoagulant effect and prevent the inflammation response caused by immune thrombosis from causing lung damage or lung failure. It can also use more heparin as the main body to promote blood circulation, more fully increase the delivery capacity of oxygen and nutrients, so as to increase the oxygen absorption capacity of the lungs and strengthen the pulmonary circulation function. But at the same time, adding dermatan sulfate and with or without chondroitin sulfate having anti-inflammatory effects can balance the anticoagulant that has a lower risk to cause bleeding while having special effects of autoimmunity repair, health care and anti-inflammatory.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A special heparinoid composition, with repair, prophylactic and anti-inflammatory effects, applied to the lungs, comprising:
at least two glycosaminoglycans, which contain 5˜95 wt % of macromolecular heparin sodium, 3˜40 wt % of low-molecule-weight heparin sodium and 5˜95 wt % of dermatan sulfate that are formulated into a solution with a mixture to water ratio of 0.1˜0.6% by weight; and
a delivery device connected to the patient's nose or mouth to deliver to said solution the lungs of the patient.

2. The special heparinoid composition, with repair, prophylactic and anti-inflammatory effects as claimed in claim 1, wherein said at least two glycosaminoglycans, which contain 85% of said macromolecular heparin sodium, 10 wt % of said low-molecule-weight heparin sodium and 5 wt % of said dermatan sulfate that are formulated into a solution with a mixture to water ratio of 0.2% by weight.

3. The special heparinoid composition, with repair, prophylactic and anti-inflammatory effects as claimed in claim 1, wherein said at least two glycosaminoglycans, which contain 10 wt % of said macromolecular heparin sodium, 40 wt % of said low-molecule-weight heparin sodium and 50 wt % of said dermatan sulfate that are formulated into a solution with a mixture to water ratio of 0.2% by weight.

4. The special heparinoid composition, with repair, prophylactic and anti-inflammatory effects as claimed in claim 1, wherein said at least two glycosaminoglycans, which contain 10 wt % of said macromolecular heparin sodium, 5 wt % of said low-molecule-weight heparin sodium and 85 wt % of said dermatan sulfate that are formulated into a solution with a mixture to water ratio of 0.3% by weight.

5. The special heparinoid composition, with repair, prophylactic and anti-inflammatory effects as claimed in claim 1, wherein said at least two glycosaminoglycans, which contain 80 wt % of said macromolecular heparin sodium, 10 wt % of said low-molecule-weight heparin sodium and 10 wt % of said dermatan sulfate that are formulated into a solution with a mixture to water ratio of 0.3% by weight.

6. The special heparinoid composition, with repair, prophylactic and anti-inflammatory effects as claimed in claim 1, wherein said at least two glycosaminoglycans, which contain 7 wt % of said macromolecular heparin sodium, 3 wt % of said low-molecule-weight heparin sodium and 90 wt % of said dermatan sulfate that are formulated into a solution with a mixture to water ratio of 0.2% by weight.

7. The special heparinoid composition, with repair, prophylactic and anti-inflammatory effects as claimed in claim 1, wherein the weight-average molecular weight of said macromolecular heparin sodium is between 5˜40 kDa, the weight-average molecular weight of said low-molecular-weight heparin sodium is between 0.4˜15 kDa, and the weight-average molecular weight of said dermatan sulfate is between 5˜80 kDa.

8. The special heparinoid composition, with repair, prophylactic and anti-inflammatory effects as claimed in claim 7, wherein the weight-average molecular weight of said macromolecular heparin sodium is 16 kDa, the weight-average molecular weight of said low-molecular-weight heparin sodium is 4.4 kDa, and the weight-average molecular weight of said dermatan sulfate is 30 kDa.

9. The special heparinoid composition, with repair, prophylactic and anti-inflammatory effects as claimed in claim 1, wherein the heparinoid further contains 0˜15 wt % chondroitin sulfate, and the weight-average molecular weight of said chondroitin sulfate is between 5˜50 kDa.

10. The special heparinoid composition, with repair, prophylactic and anti-inflammatory effects as claimed in claim 9, wherein the heparinoid contains 7 wt % of macromolecular heparin sodium, 3 wt % of low-molecule-weight heparin sodium, 88 wt % of dermatan sulfate and 2 wt % said chondroitin sulfate that are formulated into a solution with a mixture to water ratio of 0.2% by weight.

11. The special heparinoid composition, with repair, prophylactic and anti-inflammatory effects as claimed in claim 9, wherein the weight-average molecular weight of said chondroitin sulfate is between 15 kDa.

12. The special heparinoid composition, with repair, prophylactic and anti-inflammatory effects as claimed in claim 1, wherein said delivery device is selectively an ultrasonic atomizer, a metered spray inhaler or a nasal aspirator.
